(19) United States
(12) Patent Application Publication
(10) Pub. No.: US 2003/0013146 A1
(43) Pub. Date: Jan. 16, 2003

(54) Hybrid real time locating system and methodology

(76) Inventor: Jay Werb, Newton, MA (US)

Correspondence Address: John N. Anastasi, Wolf, Greenfield & Sacks, P.C., 600 Atlantic Avenue, Boston, MA, 02210, US

(21) Appl. No.: 10/138,968

(22) Filed: May 2, 2002

Related U.S. Application Data
(60) Provisional application No. 60/287,696, filed on May 2, 2001.
(60) Provisional application No. 60/350,991, filed on Jan. 23, 2002.

(51) Int. Cl. H04B 1/38; C12Q 1/68; C12M 1/34; C12Q 1/64; C12M 3/00

(52) U.S. Cl. 435/9; 435/6; 435/287.2; 455/73

(57) ABSTRACT

Hybrid Tag Device, a Hybrid Tag Protocol, a Methodology and a Hybrid System for tracking assets is disclosed. The Hybrid Tag Device comprises a Beacon Transmitter that transmits a narrow band signal, such as a UHF signal, and a wide band transmitter that transmits a wide band signal that is used to determine a distance from the Hybrid Tag Device to a Hybrid Tag Reader. The Hybrid Tag Device may also comprise a wide band signal receiver to receive a wide band signal transmitted by the Hybrid Tag Reader, which in response thereto transmits the wide band signal. In addition, the Hybrid System may comprise a sign post device that transmits an interrogation signal to the Hybrid Tag Device to enable the Hybrid Tag Device to transmit the narrow band signal.

### RELATED APPLICATIONS

This application claims priority under 35 U.S.C. § 119(e) to U.S. Provisional Application Serial No. 60/287,296 entitled “A Dual Technology RTLS Tag,” filed on May 2, 2001, and U.S. Provisional Application Serial No. 60/350,991, entitled “Hybrid Technology RTLS Tags,” filed on Jan. 23, 2002, which are herein incorporated by reference in their entirety.

### BACKGROUND OF THE INVENTION

1. Field of the Invention

The invention relates to a Hybrid Tag Device and protocol that can be used in the system to track assets. In particular, the Hybrid Tag Device comprises a narrow band transmitter that transmits a narrow band signal announcing the Hybrid Tag and a wide band transmitter that transmits a wide band signal that can be used to determine a location of the Hybrid Tag Device.

2. Description of the Related Art

In the modem enterprise, efficient resource management is a crucial step on the path to meeting business objectives. One key aspect of efficient resource management is determining the location and status of resources such as equipment, vehicles, inventory, and personnel. To this end, a number of vendors have developed Real Time Locating System (RTLS) solutions for asset tracking and data communications.

A RTLS is designed to track the locations of specially designed Tag transmitting and/or receiving devices, as known to those of skill in the art, which can be attached to assets or worn by persons, as they move through a facility. Generally, information obtained via signals sent between the Tags and installed infrastructure, such as Tag Readers, may be used to determine the location of resources within a facility. Determining a location of the Tags as used herein may indicate a general area in which a Tag is located, a precise location of the Tag (e.g., 2 or 3 dimensional coordinates of the Tag relative to a reference point), a direction in which the Tag is located relative to a reference directional point, or any other suitable indication of Tag location. For example, signal time of flight (TOF) from a Tag to a receiver may be used to determine the distance between a tagged asset and a receiver. If the receiver is installed in a known location within the facility, then the location of the tagged asset within the facility may be estimated.

Signals sent between the Tags and the infrastructure may also be used to send and receive data communications. For example, a Tag attached to an asset may be designed to obtain information from a sensor, attached to the asset. The Tag may incorporate data obtained from the sensor in its communications with the installed infrastructure. As another example, the infrastructure may send commands to the Tag, such as instructions to activate a particular equipment function or to initiate data gathering from attached sensors.

Although RTLS solutions may share certain similarities of function, these systems may vary in their implementation. For example, ranging (i. e., distance measurement) may be accomplished using signal time of flight (TOF), signal strength, or some other metric. Communications within the system may utilize various signals and devices. Even systems that use the same signals, such as radio signals, may differ in their use of various frequency bands for communications.

Equipment for an installation of RTLS asset location system can in some cases be expensive, and when the total number of assets to be tracked is small, the cost per asset may be prohibitively high. In addition, a facility wishing to add an asset tracking resource to its operations may already have one or more wireless communication systems installed and operating within the facility. Thus, adding a separate wireless tracking system to the facility may be cumbersome, interfere with other communications, and/or require unwanted additional expense.

Certain RTLS solutions may utilize microwave bands, such as 2400-2483 MHz and 5725-5875 MHz, both for ranging and for low bandwidth data communications. Due to the availability of high bandwidth used for direct sequence spread spectrum (DSSS) communication, these frequency bands are typically well suited to ranging applications, such as using signal time of flight (TOF) to estimate the distance from a tagged asset to a receiver. However, Tags using microwave bands may be subject to various types of cross-interference with high bandwidth data communication systems, such as wireless local area networks (WLANs). As modem facilities may utilize WLANs for at least some of their data communications, this cross-interference, if not mitigated, may affect the performance of both the RTLS system and/or the WLAN.

In contrast, other RTLS systems may operate at lower frequencies, such as 303.8 MHz, 418 MHz, or 433 MHz. Such systems may generally be well suited to provide one-way low bandwidth data communication over moderate distances (in excess of 100 meters) and are typically available at lower cost and operate at low power. Systems operating in these bands may also be less subject to interference, as these bands are not well suited to high bandwidth communication and are therefore less frequently used. One limitation of such systems is that they are typically not well suited to ranging applications, with signal strength typically being used to estimate the distance from a tagged asset to a receiver.

RTLS systems operating in the lower frequency bands, such as UHF bands, may involve lower equipment costs than their microwave counterparts. Both Tags and Tag Readers operating in these frequencies may be simpler and less expensive than their more sophisticated microwave counterparts. This is, in part, because available components enable the simplified design currently available and used by manufacturers of such Tags. RTLS systems operating in UHF bands or other narrowband frequencies may also achieve greater simplicity in part due to simpler functionality than that in microwave counterpart systems. For example, some UHF Tags simply announce their presence, allowing a Tag Reader to determine that a particular tagged asset is present in range of the reader but not providing information to determine the exact location of that tagged asset. Such UHF Tags may incorporate a simple design using widely available components.

In contrast, microwave Tags are typically designed to support ranging. Designers of ranging Tags often need to develop custom ASICs to achieve a reasonable cost. However, this may add to the complexity and cost of microwave RTLS systems as compared to their UHF counterparts. The increased availability of appropriate off-the-shelf microwave components, such as parts designed to support the Bluetooth standard in the 2440 MHz band, may at some point make it feasible to build inexpensive Tags in higher frequency bands. For example, vendors have designed Tags that operate in the 915 MHz band with functionality similar to the UHF products described above. However, the existence of these products does not alter the basic fact that Tags capable of ranging, regardless of their frequency of operation, tend to involve sophisticated and specific technology that tends to increase Tag and reader cost.

### SUMMARY OF THE INVENTION

A Hybrid technology Tag Device and protocol that incorporates the features of the lower frequency band RTLS systems and Hybrid higher frequency RTLS systems is desirable.

According to one embodiment of a Hybrid Tag Device and protocol of this disclosure, a Hybrid Tag Device comprises a Beacon Transmitter that transmits a first signal in a first frequency range comprising a Baseline Tag Datagram comprising a Tag ID. The Hybrid Tag Device also comprises a Local Positioning System (LPS) transmitter that transmits a second signal in a second frequency range for determining a range to the Hybrid Tag Device, and an enabling device that enables the Beacon Transmitter to transmit the first signal. In another embodiment, the Hybrid Tag Device may also comprise a LPS receiver for receiving a third signal in a third frequency range.

According to one embodiment of a hybrid system according to this disclosure, the above described Hybrid Tag Device can be used in a system for tracking assets. The system further comprises at least one Hybrid Tag Reader coupled to at least one antenna device for receiving the first signal and the second signal transmitted by the Hybrid Tag Device. According to another embodiment, the system may also comprise at least one sign post that transmits an interrogation signal, and the Hybrid Tag Device can further comprise a second receiver to receive the interrogation signal. In this embodiment, the enabling device can be configured to enable the Beacon Transmitter to transmit the first signal in response to receipt by the Hybrid Tag Device of the Interrogation signal.

According to one embodiment of a Hybrid Tag Protocol according to this disclosure, the Hybrid Tag Protocol comprises a Baseline Tag Datagram and can also comprise an optional interrogation signal portion and/or an optional ranging signal portion.

According to one embodiment of a methodology of this disclosure, a method for tracking an asset comprises transmitting with a Hybrid Tag Device coupled to the asset, a first signal in a first frequency range comprising a Baseline Tag Datagram that comprises a Tag ID. In addition, the method comprises transmitting with the Hybrid Tag Device a second signal in a second frequency range for determining a range to the asset. According to another embodiment of the method, the method may also comprise an act of receiving with the Hybrid Tag Device a third signal in a third frequency range.

According to another embodiment of the method, the above-described method may be used to track an asset and may further comprise receiving, with at least one Hybrid Tag Reader coupled to at least one antenna, the first signal and the second signal transmitted by the Hybrid Tag Device.

For any of the herein described Hybrid Tag Device, Hybrid Tag Protocol, Methodology and System, it is to be appreciated that the first signal can be used by the Hybrid Tag Device to transmit its Tag ID and to notify a Hybrid Tag Reader of its presence. In addition, the second signal transmitted by the Hybrid Tag Device can be used by the Hybrid Tag Reader to determine a range of the Hybrid Tag Device from the Hybrid Tag Reader. In addition, for some embodiments, the third signal can be transmitted by the Hybrid Tag Reader and received by the Hybrid Tag Device, and in response thereto the Hybrid Tag Device can transmit the second signal, so as to precisely determine a range from the Hybrid Tag Device to the Hybrid Tag Reader. Further, according to some embodiments, an interrogator device can transmit an interrogator signal to the Hybrid Tag Device to activate the Hybrid Tag Device when the Hybrid Tag Device is in a vicinity of the interrogation device.

### BRIEF DESCRIPTION OF THE DRAWINGS

The accompanying drawings, which are herein incorporated by reference, are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates one embodiment of a Baseline Tag Datagram according to this disclosure;

FIG. 2 illustrates one embodiment of a Hybrid Tag Datagram and Protocol according to this disclosure;

FIG. 3 illustrates one embodiment of a LPS Tag and LPS reader according to this disclosure;

FIG. 4 illustrates one embodiment of a method for detecting a presence of a Hybrid Tag Device and determining a range to the Hybrid Tag Device according to this disclosure;

FIG. 5 illustrates one embodiment of a LPS transceiver portion of a Hybrid Tag Device according to this disclosure;

FIG. 6 illustrates one embodiment of a Hybrid Tag Device according to this disclosure;

FIGS. 7a, 7b and 7c together illustrate one embodiment of a hybrid system, methodology, and protocol according to this disclosure; and

FIG. 8 illustrates alternative embodiments of a Hybrid Tag Datagram according to this disclosure.

### DETAILED DESCRIPTION

Before any embodiments of the invention are explained in detail, it is to be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

Because of the differences in functionality and cost between higher frequency band Tags that support ranging and lower frequency Tags that simply announce their presence, this specification distinguishes between these two types of Tags. For the purposes of this description, Tags capable of announcing their identities, and possibly transmitting a small amount of data, will be referred to as “RTLS Beacons,” while Tags supporting ranging will be referred to as “Local Positioning System (LPS) Tags. ” It is to be understood that the term LPS may comprise any type of ranging such as time of flight ranging between a Tag and a reference point, triangulation determination of a Tag location between the Tag and a plurality of reference points, proximity ranging based on the proximity of a Tag to a Tag Reader, and any other ranging used by those of skill in the art. It is also to be understood that according to this disclosure, a RTLS Beacon reader is any reader known to one of skill in the art that is compatible with RTLS Beacons. Further, it is to be understood that a LPS reader is any reader known to one of skill in the art that is compatible with LPS Tags.

In this disclosure, there is described a hybrid Tag device, system, protocol and methodology for taking advantage of the benefits of both types of Tags, RTLS Beacons and LPS Tags, that enables decreasing the cost of employing the two different types of systems in parallel. There is described a Hybrid RTLS Tag and methodology solution that provides the broad locating capabilities and the low bandwidth data communications capabilities of RTLS Beacons and that also provides the option for utilizing the ranging capabilities of LPS Tags. There is described a hybrid Tag device and methodology that combines the functions of RTLS Beacons and LPS Tags within a single Tag design and protocol.

A Hybrid RTLS Solution

According to one embodiment of a system according to the invention, the two aforementioned types of Tags, RTLS Beacons and LPS Tags, are utilized together within a single application. For example, a large number of assets can be tagged with relatively inexpensive RTLS Beacons, and an entire facility can be covered with relatively inexpensive RTLS Beacon Readers. The RTLS Beacon Readers allow a user of the system to track approximate RTLS Beacon/asset location, based on RTLS Beacon Reader proximity and signal strength. This capability may be sufficient for most assets in most locations within the facility. However, in certain parts of the facility or for certain assets, it may be desired to locate assets with greater precision. In these parts of the facility, costlier LPS Readers can be installed, and LPS Tags may be placed on assets that are to be tracked with greater precision. The LPS Tags provide more accurate location measurement based on known techniques, such as, signal time of flight (TOF) measurements.

In one application, a majority of assets and a majority of areas can be provided with RTLS Beacons. However, such application may also include critical assets and certain critical areas that are candidates for LPS Tags and LPS Readers. It is also to be appreciated that an entire facility may be provided with RTLS Beacon Readers in order to assist in locating lost assets. It is further to be appreciated that various modifications, applications, and derivations can be provided to the herein described Hybrid Tag Device, methodology and system, and for the sake of brevity, all such variations are not herein explicitly described. However, it is to appreciated that such variations are intended to be within the scope of this disclosure and that the scope of this disclosure is not limited except by the claims as appended hereto. For example, certain facilities may have a need for a majority of LPS Tags, or other facilities may have no need or a need for a small number of LPS Tags and LPS Readers.

One example of a use of the Hybrid Tag Device, methodology and system of this disclosure is that key subassemblies may be tracked through the manufacturing process within a limited area of the facility, where the exact location of the subassembly triggers ordering of parts, clocking operations, and so forth. For example, in an aerospace manufacturing environment, a large number of assets may be tagged with relatively inexpensive RTLS Beacons, and the entire expanse of indoor and outdoor space may be covered with relatively inexpensive RTLS Beacon Readers. This may be sufficient for determining whether a particular large piece of equipment is, for example, in Building A, B, or C. In such facilities equipped with the hybrid Tags and system of this disclosure, once the location of the piece of equipment has been narrowed down to a general vicinity, such as a single building or an area within a building, it may be a simple matter to search for an asset within that location. However, in a portion of the facility dedicated to aircraft assembly, it may also be desirable to track certain equipment or subassemblies through the assembly process in order to keep the process moving smoothly. In this part of the facility, LPS Readers may be installed, and LPS Tags may be associated with parts or process-driving equipment.

Another example is in a hospital setting, where it may be useful to know the general location of some patients or assets, such as whether a patient is currently in a particular ward or taking a walk through another part of the hospital. Thus, some patients can be provided with and may wear RTLS Beacons, and RTLS Beacon Readers may be used to cover some areas of the hospital. However, a majority of patients and/or assets may require increased monitoring and those patients or assets may be equipped with LPS Tags, and the hospital may be covered by LPS Tag Readers to allow patients to be precisely located within the hospital.

Thus, according to one embodiment of a system of this disclosure, RTLS Beacons may be placed on large numbers of assets, and relatively simple RTLS Beacon Readers may be used for broad coverage where pinpoint accuracy is not required for locating assets. To provide supplementary functionality, LPS Tags may be placed on a subset of the assets, and specialized LPS Tag Readers may be used in limited areas where asset location determination is to be more precise. It is to be understood that although the above description refers to RTLS Beacons, RTLS Beacon Readers, LPS Tags and LPS Tag Readers as separate transmitting and reading devices, that according to some embodiments of a Hybrid Tag Device, methodology and system of this disclosure, the RTLS Beacons and LPS Tags are to be combined into a Hybrid Tag Device, Hybrid protocol, and an RTLS Beacon Reader and LPS Tag Reader are combined into a single Tag Reader device.

Therefore, according to some embodiments of this disclosure it may be desirable to utilize a single Tag design and protocol, incorporating both (a) an RTLS Beacon capability, typically in the UHF band or other narrowband frequency; and (b) an optional LPS Tag capability, typically in the microwave band. It is to be appreciated that various modifications, applications, and derivations can be provided to the herein described Hybrid Tag Device, methodology and system, and for the sake of brevity, all such variations are not herein explicitly described. However, it is to appreciated that such variations are intended to be within the scope of this disclosure and that the scope of this disclosure is not limited except by the claims as appended hereto.

For example, a hybrid Tag device, methodology and system may utilize bands other than UHF and microwave. A Hybrid RTLS Tag device according to one embodiment comprises a core that includes a simple RTLS Beacon incorporating an identification capability. Such a Tag may periodically transmit its ID and perhaps a few bytes of additional information. It is to be appreciated that such transmission may be modulated, for example, amplitude modulated (AM, such as Manchester or pulse position encoded), frequency modulated (FM) or phase modulated. Such beacon transmissions may be asynchronous, such as on a periodic or randomized schedule. Alternatively, such beaconing may be in response to an interrogation signal as will be discussed herein.

For a subset of Tags that also support LPS Tag functionality, a short energy pulse may be transmitted by the hybrid Tag device, for example at some fixed time offset in relation to the beaconing signal, such as immediately following the beaconing signal, or in response to an interrogation signal. The transmitted pulse may be a direct sequence spread spectrum (DSSS) radio signal, or the pulse may be emitted by a transponder, such as a 2.4 or 5.8 GHz transponder. Alternatively, for applications where extremely high resolution is desired, the pulse may be an ultrasonic signal or an ultra wide band (UWB) signal. It is to be appreciated that various modifications, applications and derivations, can be provided to the herein described Hybrid Tag Device, and for the sake of brevity, all such variations are not herein explicitly described. However, it is to appreciated that such variations are intended to be within the scope of this disclosure and that the scope of this disclosure is not limited except by the claims as appended hereto.

Datagram for a Hybrid RTLS Tag

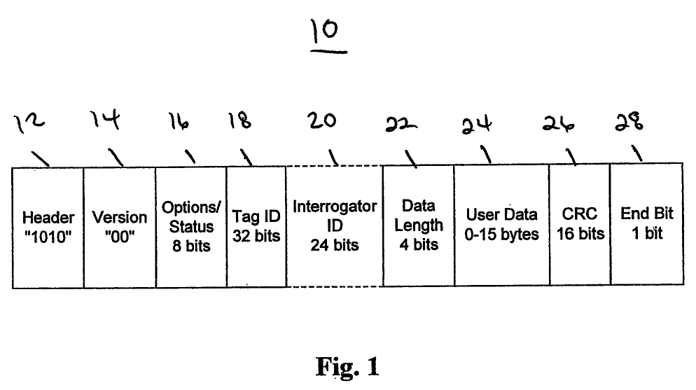

As noted herein, a Hybrid RTLS Tag may comprise a core that includes a simple RTLS Beacon that transmits a baseline Tag signal comprising a Tag ID. All Hybrid RTLS Tags may include this RTLS Beacon, with additional capabilities available on an optional basis. In an illustrative embodiment, the baseline Hybrid RTLS Tag configuration may be an RTLS Beacon that transmits a standard packet of information, called a Baseline Tag Datagram, on a schedule determined by the Tag (either periodic or asynchronous). For example, the Hybrid RTLS Tag may wake up every five seconds and transmit its Baseline Tag Datagram. Alternatively, if the Hybrid RTLS Tag is attached to a sensor, the Tag may wake up, for example, every five minutes by default, but transmit more frequently if the sensor data of the sensor to which the Hybrid RTLS Tag is coupled is changing. For example, the sensor may be a motion detector within or coupled to the Hybrid RTLS Tag, and the Hybrid RTLS Tag may change its transmission rate, typically resulting in the Hybrid RTLS Tag transmitting more frequently, when motion is detected. A Hybrid RTLS Tag may provide a Baseline Tag Datagram 10 as shown in FIG. 1. It is to be appreciated that various modifications, applications, derivations, can be provided to the herein described Baseline Tag Datagram, and for the sake of brevity, all such variations are not herein explicitly described. However, it is to appreciated that such variations are intended to be within the scope of this disclosure and that the scope of this disclosure is not limited except by the claims as appended hereto.

As shown in FIG. 1, one embodiment of the Baseline Tag Datagram 10 starts with a Header 12. The Header is used to signal the start of the Baseline Tag Datagram to the Tag Reader. The Header may also enable a Tag Reader to calibrate the Tag's bit rate by measuring the time elapsed between bit transitions in the known header bit pattern. The Baseline Tag Datagram may also comprise a Version number field 14 that enables the designer to modify the Datagram format in future product versions. The Baseline Tag Datagram may also comprise Options/Status bits 16 that contain information about the Tag, such as low battery indicator status, motion detector status, interrogator status, and/or ranging options that may be supported. The Baseline Tag Datagram may also comprise Tag ID 18 which is a unique identifier for the Tag, and is typically set by the Tag manufacturer. The Baseline Tag Datagram may also comprise Interrogator ID field 20 which is optional and may be used to repeat the identification code for an interrogator or signpost device, as further described herein. The Baseline Tag Datagram may also comprise Data Length field 22 which informs a Tag Reader that the Datagram includes User Data 24, and the length of the user data field. If no User Data is included, the Data Length field is zero and the User Data field is omitted. The Baseline Tag Datagram may also comprise (CRC) cyclic redundancy code field 26, which provides a check on the preceding data. The Baseline Tag Datagram may also comprise an End Bit 28 which is available to support shutdown of the Tag's radio.

It is to be appreciated that the Tag Datagram of FIG. 1 illustrates some general functions that may be supported by the Baseline Tag Datagram. However, various modifications, alterations and implementations exist. For example, in an implementation using pulse position encoding, the data may be encoded as a “fingerprint,” not necessarily as a series of fields. Similarly, the Tag ID may be a different length, User Data may not be supported, and the like.

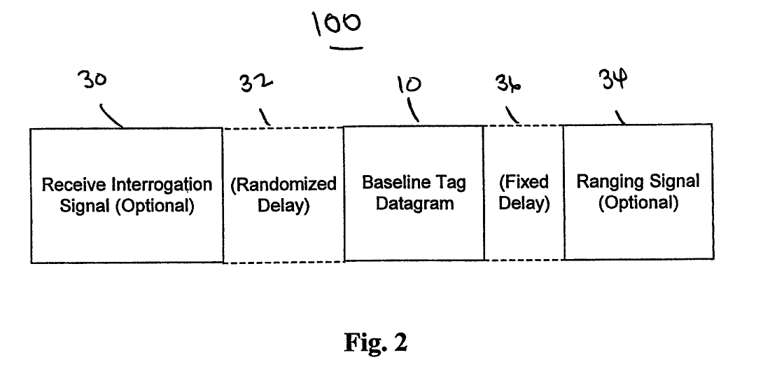

FIG. 2 illustrates a Hybrid Tag Datagram 100 that may be provided by a Hybrid RTLS Tag of this disclosure. The Hybrid Tag Datagram comprises the Baseline Tag Datagram 10 plus two optional sections 30, 34. It is to be appreciated that a Hybrid Tag Datagram according to this disclosure is a communication protocol and that the optional sections 30, 34 of the Hybrid Tag Datagram are optional signals that comprise the Hybrid Tag Datagram.

Prior to the Baseline Tag Datagram 10, the Hybrid Tag Datagram may comprise an interrogation signal section 30. The Hybrid RTLS Tag may comprise an interrogation signal receiver that can detect an Interrogation Signal 30 transmitted by an Interrogation device or signpost as known to one of skill in the art. The Interrogation Signal may comprise various forms, depending on the application. For example, the Interrogation Signal 30 may be a local activation signal, such as an inductive, RF, ultrasonic, or infrared signal provided by a signpost, for example, at a doorway. The interrogation signal may include an identification code for the interrogation device and may comprise some additional information. The Hybrid RTLS Tag may receive and repeat the interrogation device's identification code in the Baseline Tag Datagram's Interrogator ID field 20, to indicate that the Tag has passed a certain boundary associated with that interrogation device.

Alternatively, the Interrogation Signal 30 may be a signal provided by an Interrogator and addressed to a specific Tag, such as a request for the specific Tag to respond. This interrogation signal may be a short-range signal, such as from an inductive or infrared emitter, or a longer-range RF signal, such as at 13.56 MHz, 27 MHz, or 915 MHz signal. Alternatively, the Interrogation Signal may be a group page. For example, an interrogation signal may be broadcast by an interrogation device to all Tags of a designated type. If a Tag is in the designated group, it may respond.

For any of the herein described interrogation signals, the Hybrid RTLS Tag may respond to an interrogation signal received by the Hybrid RTLS Tag, after a randomized delay 32 to avoid packet collisions with other Hybrid RTLS Tags in the group, with transmission of the Baseline Tag Datagram 10 by a RTLS Beacon type transmitter. It is to be appreciated that various modifications, applications, and derivations can be provided to the herein described Hybrid Tag Device, methodology and system, and for the sake of brevity all such variations are not herein explicitly described. However, it is to appreciated that such variations are intended to be within the scope of this disclosure and that the scope of this disclosure is not limited except by the claim as appended hereto. For example, a Hybrid RTLS systems supporting a group page may include a field within the Hybrid Tag Datagram for the Tag Reader to acknowledge a response (not shown in FIG. 2), thus providing for suppression of the Hybrid RTLS Tag's continued response to the group page. In addition, it is to be appreciated that the Interrogation Signal is optional. The Hybrid RTLS Tag may transmit a Tag Datagram in response to receipt of an Interrogation Signal. Alternatively, the Hybrid RTLS Tag may transmit a Tag Datagram spontaneously or periodically based on a timer or as enabled by a sensor device such as a temperature or motion sensor.

According to one embodiment of the disclosure, the Hybrid Tag Datagram 100 ends with a Ranging Signal 34. The Ranging Signal is used to support more accurate location or ranging of the Hybrid RTLS Tag than may be possible based from the Baseline Tag Datagram 10 alone. One example of a Ranging Signal is an RF signal provided by an RF radio transponder, to be described in more detail herein. Another example of a Ranging Signal may be a DSSS signal or an Ultra Wideband (UWB) signal. With this additional capability of a Hybrid RTLS Tag and Tag Datagram, a network of Tag receivers may determine the Hybrid RTLS Tag's location using, for example, Time Difference of Arrival (TDOA). Alternatively, an UWB signal may be used with an UWB transponder, whereby a received UWB interrogation signal from a UWB interrogator prompts a precisely timed UWB signal response by the Hybrid RTLS Tag. It is to be appreciated that for this embodiment precise timing intrinsic to UWB designs can be provided by structure, as known to one of skill to provide an UWB response signal to be used for ranging. Yet another example of a Ranging Signal may be an ultrasonic pulse or series of pulses. For example, the system may clock the time that an ultrasonic pulse is received and compare it to the time that the Baseline Tag Datagram is received. The difference in time may be used to determine the distance between the Tag and an ultrasonic receiver of the system.

It is to be appreciated that the Ranging Signal 34 portion of the Hybrid Tag Datagram 100 is optional. For example, a low-cost Baseline Tag may be provided without a ranging capability. Thus, a system according to this disclosure may comprise a majority of low cost Baseline Tags that do not comprise a ranging capability and are simply RTLS Beacons, and a minority of Tags may be provided as Hybrid RTLS Tags comprising an RTLS Beacon that transmits a Baseline Tag Datagram as well as an LPS transmitter that provides a ranging signal. In such a system, a facility will contain a majority of RTLS Beacon Readers and a minority of LPS Readers, such as already been described herein. It is to be appreciated that one embodiment of a Hybrid Tag comprises a RTLS Beacon Transmitter and a LPS Transmitter. However, another embodiment of a Hybrid Tag can comprise a LPS receiver for receiving a ranging interrogation signal which can be coupled to the LPS transmitter to re-transmit the ranging interrogation signal, for example, at a different frequency such as disclosed in U.S. Pat. No. 6,150,921, herein incorporated by reference and commonly assigned U.S. application Ser. No. 09/791,037, herein incorporated by reference. It is to be appreciated that various modifications, applications, derivations of a Hybrid RTLS Tag can be provided and for the sake of brevity, all such variations are not herein explicitly described. However, it is to appreciated that such variations are intended to be within the scope of this disclosure and that the scope of this disclosure is not limited except by the claims as appended hereto.

According to one embodiment of this disclosure, a Ranging Signal 34 may include a pulse or a series of pulses used to support location determination. A Tag Reader may infer its connection to a specific Tag entirely by timing; that is, since the Ranging Signal may occur at a particular time offset 36 from a transmitted Baseline Tag Datagram 10, it may be assumed that a received Ranging Signal belongs to a particular Tag. For example, if it is known that a Ranging Signal is always sent by a Tag at a fixed delay of time t 36 after the Baseline Tag Datagram 10 is sent by that Tag, then a received Ranging Signal 34 may be assumed to have originated with whichever Tag sent a Baseline Tag Datagram at t amount of time prior to the receipt of that Ranging Signal.

According to another embodiment, it is also possible to modulate some data within the Ranging Signal 34. For example, a few bits can be used to modulate the Ranging Signal and may be used as a checksum on the Baseline Tag Datagram 10 to confirm that the Baseline Tag Datagram and Ranging Signal originated from the same Tag. It is to be appreciated that the few bits may also be expanded to a full CRC. Alternatively, at least Tag ID bits and even the entire contents of the Baseline Tag Datagram may be sent redundantly as a Modulated Ranging Signal. For example, a full Baseline Tag Datagram embedded in the Ranging Signal may be appropriate in cases where an existing ranging system with its own Tag Datagram is combined with a baseline RTLS Beacon system.

One advantage of the Hybrid Tag Device, Hybrid Tag Datagram, Methodology and System of this disclosure is that the combining an RTLS Beacon and an LPS Ranging Transmitter into a Hybrid Tag Device, and combining a Baseline Tag Datagram and an optional ranging signal into a hybrid Datagram, enables a user to select the tracking features needed for a particular application by selecting the Tag capabilities desired for a particular asset and the location capabilities desired for a particular area of coverage In this way, as has been described herein, users may opt to utilize only a limited subset of Tag functions for a majority of assets and locations, and may choose to enable more sophisticated ranging functionality for certain assets and certain locations. The Hybrid Tag device and Datagram enables these various configurations to operate together within the same architecture.

Hybrid RTLS Tag Employing a LPS Transponder

One example of an LPS Tag and system is an LPS Tag and system designed by PinPoint Corporation, which is now being sold under the name PinPoint by RF Technologies of Brookfield, Wisconsin. The LPS Tag and system is disclosed in the following commonly assigned U.S. Pat. No. 6,150,921, herein incorporated by reference, and commonly assigned U.S. application Ser. Nos. 09/694,767, 09/791,037, and 09/645,280.

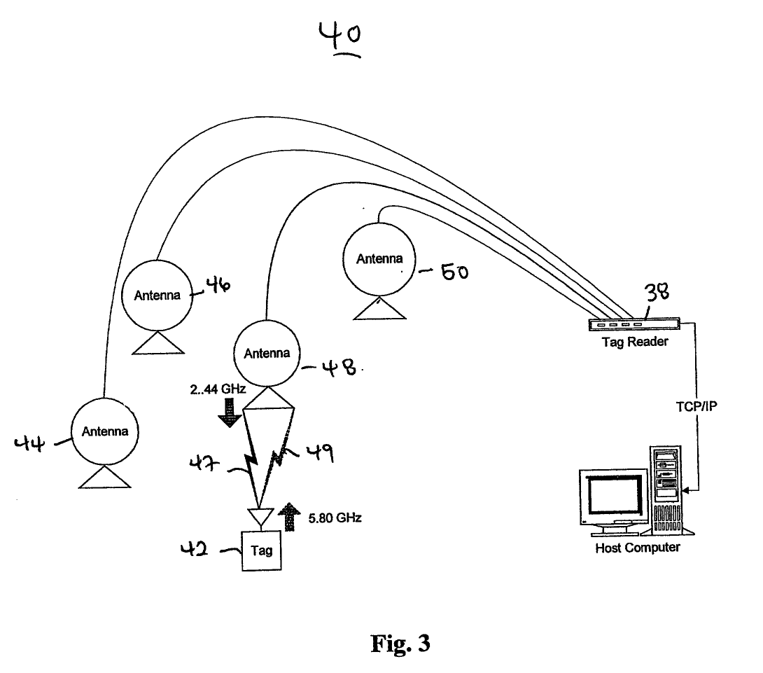

One embodiment of such an LPS system 40 is illustrated in FIG. 3: In this embodiment, a Tag Reader emits a DSSS Reference Signal 47 centered at 2442 MHz in a 2400-2483 MHz operating band. The DSSS reference Signal is transmitted by antennas 44, 46, 48 and 50, which are coupled to the Tag Reader 38, and are strategically located to cover certain areas within a facility. A Tag 42 in range of any of the antennas, receives the reference Signal 47 and up-converts the center frequency of the reference Signal from 2442 MHz to a low power return signal 49 having a 5800 MHz center frequency of a 5725-5875 MHz operating band. Tag ID information is modulated onto the return signal 49 which is transmitted by the Tag 42 back to the Tag Reader 38, via any of the antennas 44, 46, 48, 50 within range of the Tag. The Tag Reader 38 extracts the Tag ID from this return signal 49 and determines the distance of the Tag from the Tag Reader by measuring the round trip time of flight (TOF) of the transmitted DSSS signal 47 and the returned signal 49. With the multiple antennas 44, 46, 48 and 50 coupled to the Tag Reader, the Tag Reader determines the Tag distance from the multiple antennas and from multiple directions, providing information to triangulate a Tags location. The Tag 42 provides an RF mirror (frequency translation) of the Ranging Signal 47 so that the Tag Reader can locate the Tag, and the Tag modulates the Tag Reader signal to transmit the return signal 49 with the Tag information back to the Tag Reader. Since the Tag frequency converts and transponds any signal received in the 2400-2483 MHz band, a wide variety of Reference Signals may be used to locate the Tag. For example, the PinPoint LPS system utilizes 40-megachip signals. However, another Reference Signal which can be used is 11-megachip IEEE 802.11b signals.

According to one embodiment of this disclosure, the capability of the PinPoint LPS Tag 42 and system 40 may be combined with the capability of an RTLS Beacon operating in the UHF band to provide a Hybrid Tag Device and System. With this arrangement, the LPS capability of the Tag may be provided as an option so that some Tags may operate strictly as RTLS Beacons. By optional it is to be understood that the optional function such as the ranging function can be provided within a Hybrid Tag Device but need not be enabled unless a user selects the option, or optional can also mean that the Tag device may not contain this option and may simply be, for example, a RTLS Beacon.

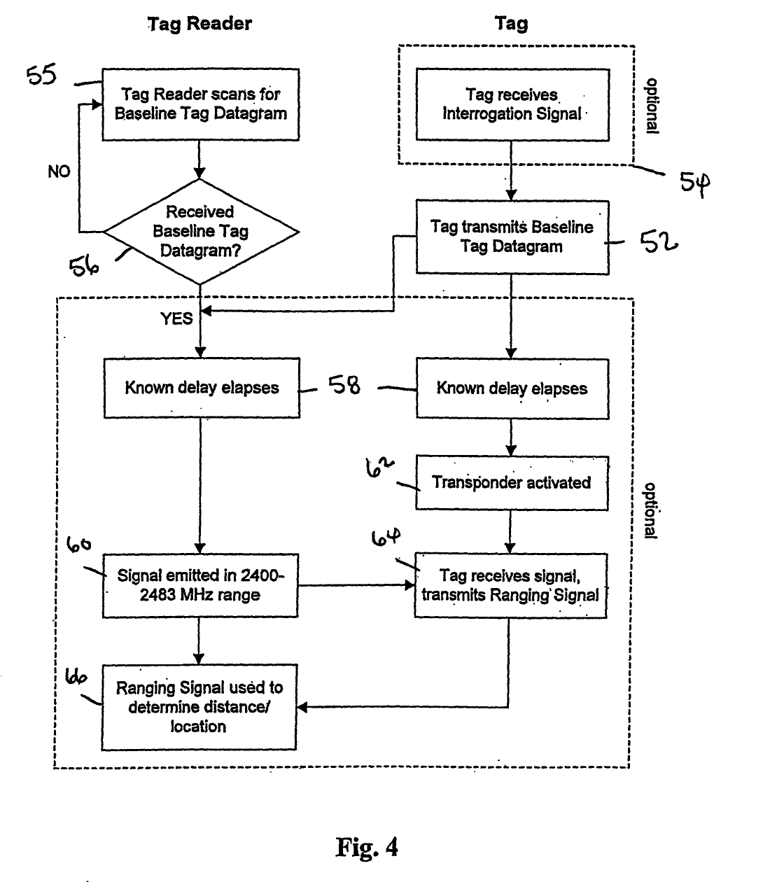

A Hybrid Tag device with an optional LPS ranging capability may operate as shown in the flow diagram of FIG. 4. A Hybrid Tag Device announces its presence (Step 52), which may be in response to receipt of an optional Interrogation Signal (Step 54), by transmitting its Baseline Tag Datagram. A Tag Reader awaits receipt of the Baseline Tag Datagram (Step 55) and upon receiving the Baseline Tag Datagram (Step 56), the Tag Reader waits until a known delay 58 elapses. Thereafter, a transponder in the Tag is activated (Step 62). The Tag Reader then emits a Reference Signal in the 2400-2483 MHz band (Step 60). Upon receiving this Reference Signal (Step 64), the Tag transmits its Ranging Signal to the Tag Reader. The Tag Reader upon receipt of the Ranging Signal (Step 66) determines the precise location of the Tag.

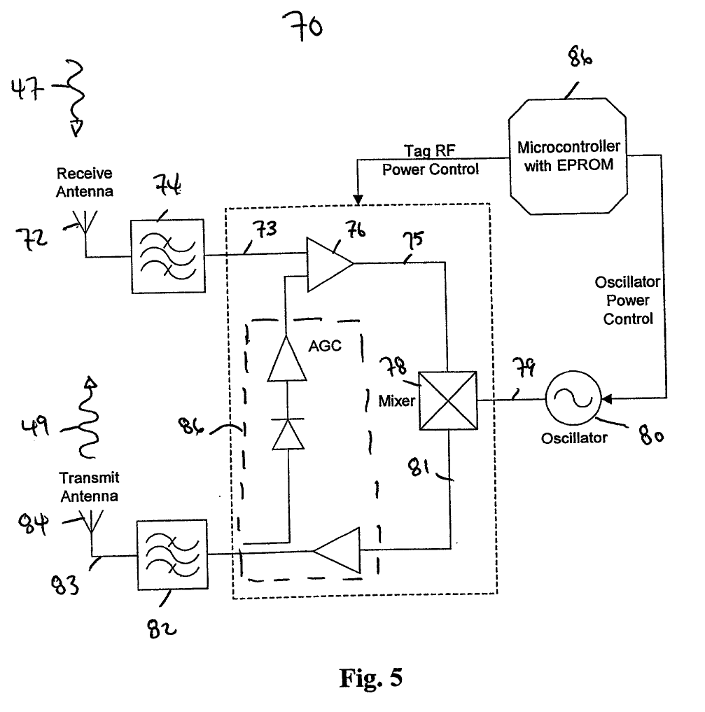

FIG. 5 is an illustrative embodiment of an LPS Tag device 70 comprising an RF transponder as described above. In this embodiment, the Tag receives an Reference Signal 47 with receive antenna 72 from a Tag Reader (not illustrated), which interrogator signal is centered at 2442 MHz or another frequency in the 2400-2483 MHz band. This interrogator signal is filtered by a filter 74, and a filtered signal 73 is provided. The filtered signal 73 is amplified by an amplifier 76 to provide an amplified signal 75. The amplified signal 75 is mixed by a mixer 78 with the output signal 79 of an oscillator, such as a 3358 MHz output signal 79, to provide a converted signal 81 of 2442+3358=5800 MHz. This converted signal 81 is filtered by a filter 82 to provide a filtered signal 83 transmitted as signal 85 by an antenna 84. In one embodiment of the LPS Tag Device 70, an Automatic Gain Control (AGC) amplifier 86 keep the operation of Tag components in an optimal operating signal range. The Tags operation is controlled by a microprocessor 86. It is to be appreciated that various modifications, alterations, and derivations of the LPS Tag, can be provided, and for the sake of brevity, all such variations are not herein explicitly described. However, it is to appreciated that such variations are intended to be within the scope of this disclosure and that the scope of this disclosure is not limited except by the claims as appended hereto.

For example, another embodiment of an LPS transponder that can be used in a Hybrid Tag Device may comprise a surface acoustic wave (SAW) device. A SAW device may be designed to retransmit a time-delayed version of a received input signal, such as a ranging interrogation signal, and/or it may modify a spectral signature of the input signal before retransmission. The SAW device can be used to provide an LPS transponder that operates similarly to the LPS transponder as illustrated in FIG. 5. In particular, the SAW device, the amplifiers and the filters can be activated for a short period of time offset from the transmission time of the Baseline Tag Datagram 10 by the Hybrid Tag Device, thus providing a signal for ranging purposes.

It is to be appreciated that according to one embodiment of this disclosure a Hybrid RTLS Tag may comprise a single case comprising shared LPS Beacon components and RTLS Beacon components, to comprise a single Hybrid Tag Device having shared resources, such as a shared microprocessor. With such an arrangement, the Hybrid RTLS Tag of this disclosure may be less expensive than a Hybrid Tag Device that is not enclosed within a single case and does not share resources. Similarly, according to one embodiment of this disclosure, a Hybrid Tag Reader of this disclosure may comprise shared components of an LPS Reader and a RTLS Beacon Reader. It is to be appreciated that various modifications, alterations and derivations of the Hybrid Tag Device and Hybrid Tag Reader, can be provided, and for the sake of brevity, all such variations are not herein explicitly described. However, it is to appreciated that such variations are intended to be within the scope of this disclosure and that the scope of this disclosure is not limited except by the claims as appended hereto. For example, a Hybrid Tag Reader can comprise a separate LPS Tag Reader and RTLS Tag Reader.

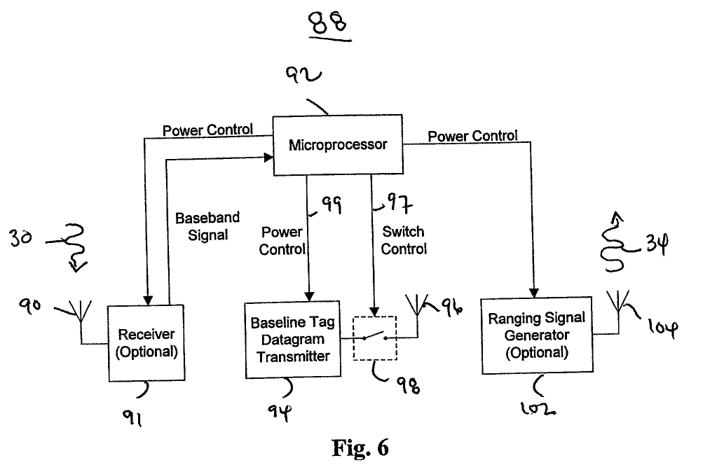

One embodiment of a Hybrid RTLS Tag 88 having a shared microprocessor 92 is illustrated in FIG. 6. In this embodiment, the Hybrid RTLS Tag comprises an antenna 90 that may receive an interrogation signal 30 as part of an optional interrogation signal receiver 91. The interrogation signal receiver converts the interrogation signal 30 to a baseband signal 91, and the data is provided to and decoded by microprocessor 92. The Hybrid RTLS Tag also comprises a transmitter coupled to antenna 96 that transmits its Baseline Tag Datagram 10. A switch 98 can be provided at an output of the Baseline Datagram transmitter and under control of switch control signal 97 provided by the microprocessor 92, which can be used to provide fast AM modulation of the Baseline Tag Datagram. It is to be appreciated that slower AM modulation of the Baseline Tag Datagram may be achieved without the switch 98, for example, through the power control signal 99 or that other forms of modulation such as FM, BPSK may be used. The Hybrid RTLS Tag also comprises an optional Ranging Signal Generator 102 that generates a Ranging Signal 34, and transmits the Ranging Signal 34 via an antenna 104. Operation of the Hybrid RTLS Tag is controlled by the shared microprocessor 92. It is to be appreciated that various modifications, alterations and derivations of the Hybrid Tag Device can be provided, and for the sake of brevity, all such variations are not herein explicitly described. However, it is to appreciated that such variations are intended to be within the scope of this disclosure and that the scope of this disclosure is not limited except by the claims as appended hereto.

For example, the Interrogation Signal 30 may be inductive, ultrasonic, RF, infrared, or of some other form of signal. The Interrogation Signal may be sent by an Interrogator or Signpost, for example, as a Tag is passing a doorway or some other location marker, and the Tag may indicate in its Baseline Tag Datagram 10, in the Interrogator ID field 20, the ID of the interrogation device from which it last received an Interrogation Signal. It is also to be appreciated that the Interrogation Signal may be broadcast to a wider area than just at a portal, and the Interrogation Signal may be broadcast to all Tags, to all Tags within a group, or to a specific Tag. Alternatively, no Interrogation Signal may be used and the Tag may broadcast its identify periodically or even spontaneously.

Thus, one advantage of the Hybrid Tag Device, Hybrid Tag Datagram, methodology and system of this disclosure is that they provide for a plurality of RTLS Beaconing capabilities and LPS Tag capabilities. For example, in lieu of a transponder-based ranging, a Hybrid RTLS Tag may incorporate a ranging capability based on TDOA, it may incorporate an ultrasonic ranging capability, or it may utilize another methodology or signal type for ranging. Thus, the shared design and protocol of the hybrid system of this disclosure yields great flexibility in implementation.

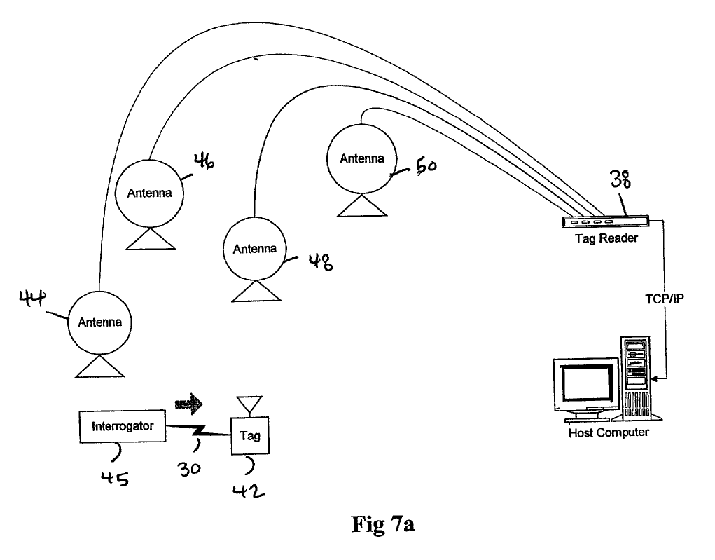

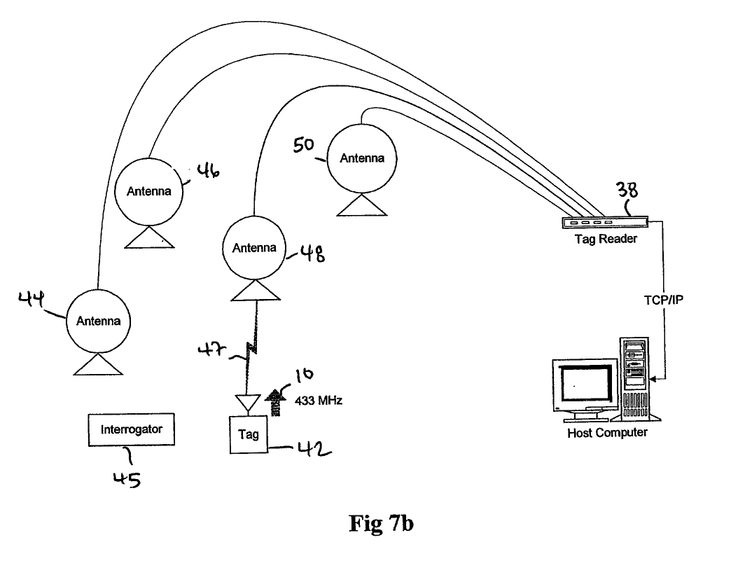

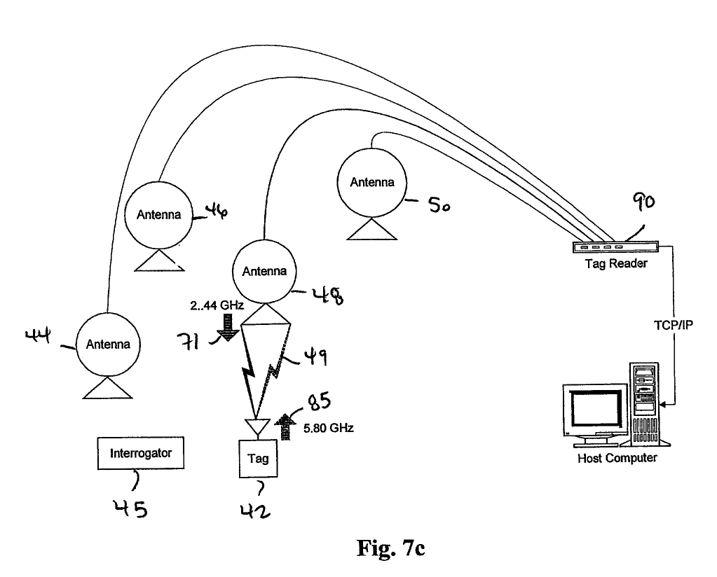

FIGS. 7a, 7b, and 7c together illustrate one possible operation of an embodiment of a Hybrid RTLS system. In FIG. 7A, a Hybrid Tag Device 42 receives an Interrogation Signal 30, such as an inductive signal, an RF signal, or an infrared signal provided by interrogator 45. The Interrogation Signal includes a unique code for the Interrogator, which in turn can be used as an indication of the location of the Tag at the time it receives the Interrogation Signal. The Interrogation Signal may also comprise commands and/or data to the be sent to the Tag.

Referring now to FIG. 7b, after a delay 32 (See FIG. 2), the Hybrid RTLS Tag 42 transmits a Baseline Tag Datagram 10, for example, on a 433 MHz carrier signal 47, which is received by a Tag Reader via antenna 48. It is to be appreciated that although the Hybrid Tag Datagram is described as being transmitted in the UHF band of 433 MHz, any inexpensive and power-efficient radio with sufficient range would fulfill the same function. The Baseline Tag Datagram 10 includes, among other things, the unique identifier 18 of the Tag and the identifier of the Interrogator 20, as described with respect to FIG. 2.

Referring now to FIG. 7c, after a delay 36 (See FIG. 2), the Hybrid RTLS Tag briefly activates a transponder, which receives the 2442 MHz Interrogation Signal 71 and converts the 2442 MHz Interrogation Signal to a 5800 MHz converted signal 49, as previously described in connection with FIG. 5. During this brief period of activation of the transponder, the Hybrid Tag Reader 90 emits a DSSS signal 71 centered at 2442 MHz, and looks for a transponded response signal 71 centered at 5800 MHz, and uses this transponded signal to determine the distance between the antenna 48 and the Hybrid RTLS Tag.

For some applications of the Hybrid RTLS system, a mobile version of the Tag Reader is desirable, particularly in a handheld package. Users of prior art handheld Beacon Tag readers use signal strength to locate a Beacon tag. If a Beacon tag transmits every five seconds, the handheld Beacon Tag Reader may display the tag signal strength as, for example, a bar that increases in size every five seconds as the user moves closer and the signal grows stronger. This approach may be used to find Beacon tags, but it is far from ideal. For example, signal strength is not a very reliable indication of distance from the tag, and it is not unusual for signal strength to decrease even as a person moves closer to the Beacon Tag. Ultimately close proximity to the Beacon tag will be reflected in high signal strength indication with the mobile Beacon Tag Reader, but the process of receiving higher signal strength—lower signal strength indications can ineffective and frustrating.

In contrast, a mobile version of a Hybrid Tag Reader according to one embodiment of this disclosure is much easier to use. A mobile Hybrid Tag Reader can be configured so that a distance to the tag is displayed to the user. As the user gets closer to the object, the user display can indicate a distance measurement based on the relatively reliable metric of time-of-flight. Objects are thus quickly and reliably found.

According to one embodiment, a mobile Hybrid Tag Reader may use 802.11b signals for ranging. Such signals may provide accuracy on the order of 10 feet, and can be generated using small, power-efficient, and inexpensive modules that are already being mass-produced for 802.11b radios. This mobile Tag Reader has another advantage in that if the mobile Hybrid Tag Reader uses an 802.11b radio for general communications, the electronics can be shared and can be used for two functions.

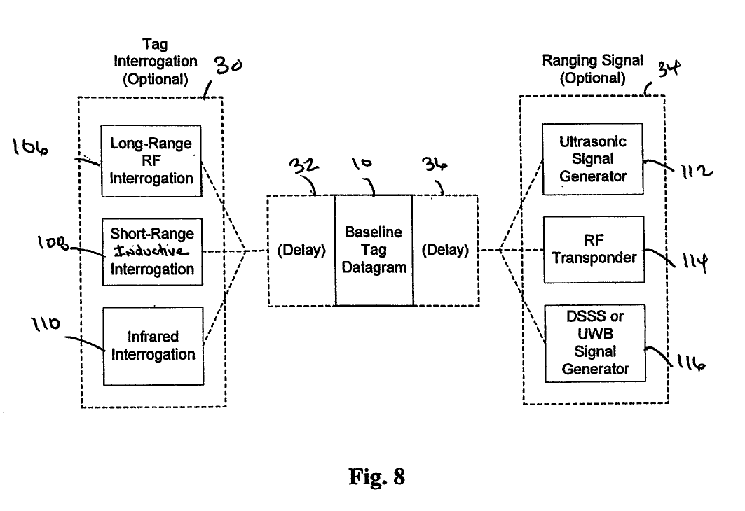

FIG. 8 illustrates an expanded Hybrid Tag Datagram 100 that illustrates some of the options that can be used for the Tag Interrogation 30 and Ranging 34 Signals. As noted herein, the Hybrid Tag Datagram comprises the Baseline Tag Datagram 10 plus two optional portions 30, 34 to support Tag interrogation and Ranging. It is to be appreciated that options for the Tag interrogation portion 30 include a long-range RF interrogation signal 106, a short-range inductive interrogation signal 108, or an Infrared Interrogation Signal 110. Options for a Ranging Signal comprise an ultrasonic signal 112, an RF or UWB transponder signal 114, or a DSSS or UWB signal 116. It is to be appreciated that the Hybrid Tag Datagram illustrated in FIG. 8 illustrate several possible embodiments of a Hybrid Tag Datagram, but that various modifications exist and are not herein described simply for the sake of brevity. However, it is to be appreciated that such variations are within the scope of this disclosure and the scope of this disclosure is not limited except by the claims as appended hereto.

To illustrate the degree of flexibility that exists with the Hybrid RTLS Tag, Hybrid RTLS Tag Datagram, the methodology and system of this disclosure, the following is an example of a system that can be used, and of a number of options from which a user can pick and choose. The system may comprise a plurality of Tags that provide a Baseline Tag Datagram. Some of the Tags can support providing Ranging Signals by, for example, a 2.4/5.8 GHz transponder. Fixed Beacon Tag Readers may be provided to cover some areas of a facility. Mobile Beacon Tag Readers may be provided to use in some areas of the facility. Some of the Tags can be adapted to receive inductive interrogation signals to indicate that a Tag is passing a particular portal or signpost. Some portals of the facility (or some of the Mobile Beacon Tag Readers) may be adapted to include inductive Tag interrogator. Some areas of the facility can be provided with LPS Tag Readers and some of the LPS Tag Readers may be supplemented by low-resolution circuitry capable of measuring distances to Tags using IEEE 802.11b signals. In addition, some of the LPS Tag Readers may be supplemented by high-resolution circuitry capable of measuring distances to Tags using 40 megachip signals. Further, some of the Mobile Beacon Tag Readers may also include the ability to measure distance to Tags. In a facility comprising the above devices, all of these options are supported by the single Hybrid Tag architecture of this disclosure, and may be selected by the user on an as-needed basis. One advantage of the Hybrid Tag Datagram, Hybrid Tag Device, methodology and system of this disclosure is Pareto cost reduction. The herein described Hybrid approach may seem to increase the Tag cost and infrastructure cost by virtue of involving multiple Tag capabilities and infrastructures. However, the hybrid approach can result in dramatic cost reduction. In an industrial application, such as in an aerospace facility, a simple RTLS Beacon such as a UHF emitter may be adequate for a majority of assets. Likewise, simple RTLS Beacon Readers such as UHF readers, sometimes in conjunction with the ability to transmit and receive with a Hybrid RTLS Tag short-range interrogation signals, may be adequate for a majority of areas covered. Since more sophisticated Hybrid RTLS Tags comprising a ranging capability may only be needed for a subset of assets, and the installation of Ranging Hybrid RTLS Tag Readers may only be needed for particular areas within a facility, this hybrid system approach can dramatically decrease the system costs.

An additional advantage of the Hybrid Tag Datagram, Hybrid Tag Device, methodology and system of this disclosure is that cross-interference such as discussed herein between an LPS Tag or LPS Tag Reader and WALN, for example in the 2400-2483 MHz band can be minimized or eliminated.

For example, in a non-hybrid system, an overwhelming majority of a Tag Reader's time may be spent waiting for an LPS Tag to transmit, or searching for an LPS Tags. For Example, the LPS Tags may operate by waking up periodically, receiving an incoming signal at 2.4 GHz transmitted by the LPS Reader and converting it to 5.8 GHz. In order for a Tag Reader to see the Tags, the Tags must be illuminated with the signal in the 2400-2483 MHz band. As a result, an LPS Tag Reader (or another device) must emit the signal in the 2400-2483 MHz band during each Tag search, in case a Tag happens to be enabled and transponding at a given moment. However such a signal transmitted by a Tag Reader may interfere with a WLAN operating in the same frequency band, in particular an IEEE 802.11b WLAN.

However, an alternate solution is provided by the Hybrid Tag Device, the Methodology and the system of this disclosure, which can search for Tags without using the 2400-2483 MHz band. One implementation of the Hybrid RTLS Tag Device and System achieves this result by first having the Tag identify itself to a Hybrid Tag Reader by emitting a Baseline Tag Datagram 10 in a non-interfering frequency band (e. g. UHF). Only after the Tag is detected by the Hybrid Tag Reader, and only when the Tag has enabled its Ranging Signal transponder (after a known delay), will the Tag Reader transmit potentially interfering energy in the 2400-2483 MHz band, and then only for the very short period of time to determine the distance from the Tag Reader to the Tag. With this arrangement, two fundamental reductions in interference result. First, potentially interfering energy may be transmitted by a Hybrid Tag Reader only when the Tag's transponder is known to be operating. The hybrid interrogator thus may not need to transmit any energy while searching for Hybrid Tags Devices. Second, the amount of time needed to illuminate the Tag with the interfering signal is reduced, since only a portion of the Hybrid Tag Datagram is involved in distance measurement.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and FIGs. are by way of example only.

What is claimed is:

1. A Hybrid Tag Device, comprising:
a Beacon Transmitter that transmits a first signal in a first frequency range comprising a Baseline Tag Datagram comprising a Tag ID;
an optional Local Positioning System (LPS) transmitter that can be enabled to transmit a second signal in a second frequency range for calculating a location of the Hybrid Tag Device; and
an enabling device that enables the Beacon Transmitter to transmit the first signal.

2. The Hybrid Tag Device as claimed in claim 1, further comprising a Reference Signal receiver for receiving a third signal in a third frequency range.

3. The Hybrid Tag Device as claimed in claim 2, further comprising a frequency translation device that operates on the third signal to frequency translate the third signal in the third frequency range to the second signal in the second frequency range.

4. The Hybrid Tag Device as claimed in claim 1, wherein the enabling device comprises a timing device that periodically enables the Beacon Transmitter to transmit the first signal.

5. The Hybrid Tag Device as claimed in claim 1, wherein the enabling device comprises a motion detector that detects a motion of the Hybrid Tag Device and enables the Beacon Transmitter to transmit the first signal.

6. The Hybrid Tag Device as claimed in claim 1, wherein the enabling device comprises a second receiver for receiving an interrogation signal and wherein the enabling device enables the Beacon Transmitter to transmit the first signal in response to receipt of the interrogation signal.

7. The Hybrid Tag Device as claimed in claim 6, wherein the second receiver is a radio frequency (RF) receiver.

8. The Hybrid Tag Device as claimed in claim 6 wherein the second receiver is an inductive signal receiver.

9. The Hybrid Tag Device as claimed in claim 6, wherein the second receiver comprises an infrared (IR) receiver.

10. The Hybrid Tag Device as claimed in claim 6, wherein the second receiver comprises an ultrasonic signal receiver.

11. The Hybrid Tag Device as claimed in claim 1, wherein the first frequency range is a narrow band frequency range.

12. The Hybrid Tag Device as claimed in claim 1, wherein the first frequency range comprises a UHF frequency range,

13. The Hybrid Tag Device as claimed in claim 1, wherein the Beacon Transmitter comprises a modulator that modulates the first signal.

14. The Hybrid Tag Device as claimed in claim 13, wherein the modulator modulates the first signal using amplitude modulation.

15. The Hybrid Tag Device as claimed in claim 13, wherein the modulator modulates the first signal using phase-shift keyed modulation.

16. The Hybrid Tag Device as claimed in claim 13, wherein the modulator modulates the first signal using frequency modulation.

17. The Hybrid Tag Device as claimed in claim 3, wherein the third signal has a center frequency substantially in a range of 2400-2483 MHz and the second signal has a center frequency substantially in a range of 5725-5875 MHz.

18. The Hybrid Tag Device as claimed in claim 1, wherein the LPS transmitter comprises a pulsed RF transmitter.

19. The Hybrid Tag Device as claimed in claim 1, wherein the LPS transmitter comprises a UWB signal transmitter.

20. The Hybrid Tag Device as claimed in claim 1, wherein the LPS transmitter comprises a DSSS transmitter.

21. The Hybrid Tag Device as claimed in claim 1, wherein the LPS transmitter comprises an ultrasonic signal transmitter.

22. The Hybrid Tag Device as claimed in claim 1, incorporated into a system for tracking assets, the system further comprising at least one Hybrid Tag Reader coupled to at least one antenna for receiving the first signal and for receiving the second signal.

23. The system as claimed in claim 22, wherein the Hybrid Tag Device further comprises a Reference Signal receiver for receiving a third signal in a third frequency range, and wherein the at least one Hybrid Tag Reader further comprises a transmitter for transmitting the third signal.

24. The system as claimed in claim 22, further comprising a processor for determining a location of the Hybrid Tag Device from the at least one Hybrid Tag Reader based on a time of reception of the second signal by the at least one Hybrid Tag Reader.

25. The system as claimed in claim 22, wherein the Hybrid Tag Reader further comprises a cell controller and the at least one antenna comprises a plurality of antennas, and wherein the cell controller is coupled to the plurality of antennas and is configured to transmit the third signal from each of the plurality of antennas and to receive the second signal at each of the plurality of antennas, and wherein the cell controller is configured to determine a distance from the Hybrid Tag Device to each antenna that receives the second signal based on a time of reception of the second signal at each antenna device.

26. The system as claimed in claim 22, further comprising at least one signpost that transmits an interrogation signal; and
wherein the Hybrid Tag Device further comprises a second receiver to receive the interrogation signal, and wherein the enabling device is configured to enable the Beacon Transmitter to transmit the first signal in response to receipt of the interrogation signal.

27. The system as claimed in claim 26, wherein the signpost is configured to transmit the interrogation signal comprising an ID of the signpost.

28. The system as claimed in claim 26, wherein the signpost is configured to transmit the interrogation signal as a paging signal to be received by any Hybrid Tag Device within the transmission range of the signpost.

29. The system as claimed in claim 26, wherein the signpost is configured to transmit the interrogation signal as a targeted signal intended for a targeted Hybrid Tag Device.

30. The system as claimed in claim 26, wherein the signpost transmits an inductive interrogation signal.

31. The system as claimed in claim 26, wherein the signpost transmits an infrared (IR) interrogation signal.

32. The system as claimed in claim 26, wherein the signpost transmits a radio frequency (RF) interrogation signal.

33. The system as claimed in claim 26, wherein the signpost transmits an ultrasonic interrogation signal.

34. The system as claimed in claim 22, wherein the Hybrid Tag Reader comprises a Beacon receiver that receives the second signal in the second frequency range.

35. The system as claimed in claim 22, wherein the Hybrid Tag Reader is a hand-held LPS receiver that enables the person to traverse an area to receive the second signal in the second frequency range to locate the Hybrid Tag Device.

36. The system as claimed in claim 22, wherein the Hybrid Tag Reader comprises a second LPS reader to receive the second signal in the second frequency range and to determine a distance to the Hybrid Tag Device.

37. The system as claimed in claim 36, wherein the second LPS receiver determines a distance to the Tag based on a power level of the received second signal.

38. The system as claimed in claim 36, wherein the second LPS receiver comprises a pulsed RF receiver.

39. The system as claimed in claim 36, wherein the second LPS receiver comprises an ultra wide band receiver.

40. The system as claimed in claim 36, wherein the second LPS receiver comprises a direct sequence spread spectrum (DSSS) receiver.

41. The system as claimed in claim 36, wherein the second LPS receiver comprises an ultrasonic receiver.

42. The Hybrid Tag Device as claimed in claim 1, wherein the Baseline Tag Datagram further comprises a data field comprising data about an asset to which the Hybrid Tag Device is to be coupled.

43. The Hybrid Tag Device as claimed in claim 1, wherein the Baseline Tag Datagram further comprises status bits comprising information about the Hybrid Tag Device.

44. The Hybrid Tag Device as claimed in claim 43, wherein the status bits comprise information about a battery of the Hybrid Tag Device.

45. The Hybrid Tag Device as claimed in claim 43, wherein the status bits comprise information about a motion detector coupled to the Hybrid Tag Device.

46. The Hybrid Tag Device as claimed in claim 1, wherein the Baseline Tag Datagram further comprises an interrogator ID field to comprise an ID of an interrogator device.

47. The Hybrid Tag Device as claimed in claim 1, wherein the Baseline Tag Datagram further comprises a user data field to comprise data provided by a user of the Hybrid Tag Device.

48. The Hybrid Tag Device as claimed in claim 42, wherein the data field comprises data from a temperature sensor.

49. The Hybrid Tag Device as claimed in claim 42, wherein the data field comprises data from a motion detector.

50. The Hybrid Tag Device as claimed in claim 1, wherein the second signal comprises a ranging portion of a Hybrid Tag Datagram and wherein the first signal and the second signal together comprise the Hybrid Tag Datagram.

51. The Hybrid Tag Device as claimed in claim 50, wherein the Hybrid Tag Datagram further comprises an interrogation signal portion comprising an interrogation signal provided by an interrogated device.

52. The Hybrid Tag Device as claimed in claim 50, wherein the ranging portion of the Tag Datagram comprises a confirmation of the Hybrid Tag Device ID.

53. A method of tracking an asset, comprising:
transmitting with a Hybrid Tag Device a first signal in a first frequency range comprising a Baseline Tag Datagram that comprises a Tag ID; and
optionally transmitting with the Hybrid Tag Device a second signal in a second frequency range for calculating a location to the asset.

54. The method as claimed in claim 53, further comprising an act of receiving a third signal in a third frequency range with the Hybrid Tag Device.

55. The method as claimed in claim 53, further comprising an act of translating the third signal in the third frequency range to the second signal in the second frequency range.

56. The method as claimed in claim 53, further comprising an act of periodically enabling the Hybrid Tag Device to transmit the first signal.

57. The method as claimed in claim 53, further comprising an act of detecting a motion of the Hybrid Tag Device and enabling the Hybrid Tag Device to transmit the first signal in response to detection of the motion of the Hybrid Tag Device.

58. The method as claimed in claim 53, further comprising an act of receiving an interrogation signal and enabling the Hybrid Tag Device to transmit the first signal in response to receiving the interrogation signal.

59. The method as claimed in claim 58, wherein the act of receiving the interrogation signal comprises receiving a radio frequency (RF) signal.

60. The method as claimed in claim 58, wherein the act of receiving the interrogation signal comprises receiving an inductive signal.

61. The method as claimed in claim 58, wherein the act of receiving the interrogation signal comprises receiving an infrared (IR) signal.

62. The method as claimed in claim 58, wherein the act of receiving the interrogation signal comprises receiving an ultrasonic signal.

63. The method as claimed in claim 53, wherein the act of transmitting the first signal comprises transmitting a narrow band signal.

64. The method as claimed in claim 53, wherein the act of transmitting the first signal comprises transmitting a UHF signal.

65. The method as claimed in claim 53, further comprising an act of modulating the first signal.

66. The method as claimed in claim 65, wherein the act of modulating the first signal comprises amplitude modulating the first signal.

67. The method as claimed in claim 65, wherein the act of modulating the first signal comprises phase-shift key modulating the first signal.

68. The method as claimed in claim 65, wherein the act of modulating the first signal comprises frequency modulating the first signal.

69. The method as claimed in claim 55, wherein the act of translating comprises translating the third signal having a center frequency substantially in the range of 2400-2483 MHz to the second signal having a center frequency substantially in a range of 5725-5875 MHz.

70. The method as claimed in claim 53, wherein the act of transmitting the second signal comprises transmitting a pulsed RF signal.

71. The method as claimed in claim 53, wherein the act of transmitting the second signal comprises transmitting a UWB signal.

72. The method as claimed in claim 53, wherein the act of transmitting the second signal comprises transmitting a DSSS signal.

73. The method as claimed in claim 53, wherein the act of transmitting the second signal comprises transmitting an ultrasonic signal.

74. The method as claimed in claim 53, further comprising an act of receiving the first signal with at least one Hybrid Tag Reader coupled to at least one antenna device.

75. The method as claimed in claim 74, further comprising an act of receiving a third signal in a third frequency range with the Hybrid Tag Device and an act of transmitting the third signal with the at least one Hybrid Tag Reader.

76. The method as claimed in claim 74, further comprising an act of receiving the second signal with the at least one Hybrid Tag Reader and determining a location of the Hybrid Tag Device from the at least one Hybrid Tag Reader based on a time of reception of the second signal by the at least one Hybrid Tag Reader.

77. The method as claimed in claim 76, wherein the act of transmitting the third signal comprises transmitting the third signal with a plurality of antennas, and wherein the act of receiving the second signal comprises receiving the second signal with the plurality of antennas, and the act of determining the location of the Hybrid Tag Device comprises determining a disclosure from the Hybrid Tag Device to each of the plurality of antennas that receives the second signal based on a time of reception of the second signal at each antenna device.

78. The method as claimed in claim 74, further comprising an act of transmitting an interrogation signal with a sign post device, receiving the interrogation signal with the Hybrid Tag Device, and enabling the Hybrid Tag Device to transmit the first signal in response to receipt of the interrogation signal.

79. The method as claimed in claim 78, wherein the act of transmitting the interrogation signal comprises transmitting the interrogation signal comprising an ID of the sign post.

80. The method as claimed in claim 78, wherein the act of transmitting the interrogation signal comprises transmitting a paging signal to be received by any Hybrid Tag Device within a transmission range of the sign post device.

81. The method as claimed in claim 78, wherein the act of transmitting the interrogation signal comprises transmitting a targeted signal intended for a targeted Hybrid Tag Device.

82. The method as claimed in claim 78, wherein the act of transmitting the interrogation signal comprises transmitting an inductive signal.

83. The method as claimed in claim 78, wherein the act of transmitting the interrogation signal comprises transmitting an infrared (IR) signal.

84. The method as claimed in claim 78, wherein the act of transmitting the interrogation signal comprises transmitting a radio frequency (RF) signal.

85. The method as claimed in claim 78, wherein the act of transmitting the interrogation signal comprises transmitting an ultrasonic signal.

86. The method as claimed in claim 74, further comprising an act of receiving with the Hybrid Tag Reader the second signal in the second frequency range.

87. The method as claimed in claim 74, further comprising an act of receiving with a hand held receiver by a person traversing a facility, the second signal to locate the Hybrid Tag Device.

88. The method as claimed in claim 86, further comprising the act of determining a distance to the Hybrid Tad Device based on a power level of the received second signal.

89. The method as claimed in claim 86, wherein the act of receiving the second signal comprises receiving a pulsed RF signal.

90. The method as claimed in claim 86, wherein the act of receiving the second signal comprises receiving an ultra wide band (UWB).

91. The method as claimed in claim 86, wherein the act of receiving the second signal comprises receiving a direct sequence spread spectrum (DSSS) signal.

92. The method as claimed in claim 86, wherein the act of receiving the second signal comprises receiving an ultrasonic signal.

93. The method as claimed in claim 53, further comprising the act of providing the Baseline Tag Datagram with a data field to comprise data about an asset to which the Hybrid Tag Device is to be coupled.

94. The method as claimed in claim 53, further comprising the act of providing the Baseline Tag Datagram with status bits to comprise information about the Hybrid Tag Device.

95. The method as claimed in claim 94, wherein the act of providing the Baseline Tag Datagram comprises providing the status bits with information about a battery of the Hybrid Tag Device.

96. The method as claimed in claim 94, wherein the act of providing the Baseline Tag Datagram comprises providing the status bits with information about a motion detector coupled to the Hybrid Tag Device.

97. The method as claimed in claim 53, further comprising the act of providing the Baseline Tag Datagram with an interrogator ID field to comprise an ID of an interrogator device.

98. The method as claimed in claim 53, further comprising the act of providing the Baseline Tag Datagram with a user data field to comprise data provided by a user of the Hybrid Tag Device.

99. The method as claimed in claim 93, where in the act of providing the data field comprises providing the data field with data from a temperature sensor.

100. The method as claimed in claim 93, wherein the act of providing the data field comprises providing the data field with data from a motion detector.
